(19) United States
(12) Patent Application Publication
(10) Pub. No.: US 2015/0257868 A1
(43) Pub. Date: Sep. 17, 2015

(54) INTERLACED PARTICULATE FILTER

(71) Applicant: KEYSTONE HEART LTD., Caesarea (IL)

(72) Inventor: Yuval Shezifi, Haifa (IL)

(21) Appl. No.: 14/435,929

(22) PCT Filed: Oct. 14, 2013
(86) PCT No.: PCT/IL2013/050826

§ 371 Date: Apr. 15, 2015

Related U.S. Application Data
(60) Provisional application No. 61/714,401, filed on Oct. 16, 2012.

(51) Int. Cl. A61F 2/01; A61F 2/07; A61F 2/24; A61F 2/90

(52) U.S. Cl. CPC A61F 2/013; A61F 2/01; A61F 2/90; A61F 2/07; A61F 2/24; A61F 2210/0014; A61F 2002/016

(57) ABSTRACT

The device of the invention may include an interlaced portion (1) and internal inserts (17) that exit the interlaced portion, form a loop, e.g., an external member, and optionally re-enter the interlaced portion. The interlace portion may filter and/or deflect emboli or other large objects from entering protected secondary vessels, while the external member may facilitate device placement within the primary vessel. The device may further be compatible with common delivery methods used in interventional cardiology (e.g., TAVI procedures). The device may be integrated into a delivery system. In other embodiments, the device may be detachable from the delivery system. Upon deployment, the device may be positioned so as to contact the orifice of one or more secondary blood vessels to, e.g., the aortic arch. In still other embodiments, the filaments may be arranged so that the edge of the device is relatively flexible and may serve as a gasket for improved device performance within a primary vessel.

### CROSS-REFERENCE TO RELATED APPLICATION

This application claims benefit of U.S. Provisional Application No. 61/714,401 filed Oct. 16, 2012, which is hereby incorporated by reference in its entirety.

### FIELD OF THE INVENTION

The invention relates to devices and methods for blocking emboli in an aorta from entering arteries.

### BACKGROUND OF THE INVENTION

Emboli form, for example, as a result of the presence of particulate matter in the bloodstream. Vascular emboli are a major single causative agent for multiple human pathologies. It is a leading cause of disability and death.

Since emboli are typically particulate in nature, various devices, e.g., types of filters, have been proposed in an attempt to remove or divert such particles from the bloodstream. These devices may be inserted into a blood vessel prior to or during a procedure or at another time. Such devices may be inserted by way of a catheter that may be threaded through a vein or artery and into, for example, an aorta or other vessel where the device may be released from the catheter and, for example, deployed. The device may filter, deflect, or block emboli or other objects from entering into a blood supply that feeds the brain.

The devices known in the art, generally lack a suitable anchoring system, as the pulsating blood flow, aortic elasticity, and movement may all cause a device inserted into a major blood vessel to become dislodged. Furthermore, such devices may feature rigid structures that may create turbulent blood flow at certain locations such as the aortic arch, leading to decreased cerebral flow and possible activation of the closing mechanism. Additionally, the manufacture of such devices and/or structures is a complicated process because these device require multiple components to form their required elements, rather than having the ability to form the required elements from a single component.

Therefore, there is a need for a more effective and more easily manufactured device and method for protecting against particulate such as emboli.

### SUMMARY OF THE INVENTION

In one aspect, the invention features an intra-vascular device for deflecting emboli including an interlaced portion, (e.g., a filter), and at least one or two (or more) loop portions, (e.g., a portion where one or two (or more) interlaced filaments and/or internal inserts exits and re-enters the interlaced portion). This filter may include a plurality of filaments interlaced together including an edge. These loops can be further configured, e.g., twisted, so as to form an extern at member and can include structural filaments. In some cases, all, a portion, or a subset of the exited interlaced filaments and/or internal inserts, (e.g., one wire, at least two wires) may not re-enter the interlaced portion thereby maintaining axial strength in the device.

In the devices of the invention, the internal insert can be capable of providing structural support and further be capable of providing functionality, e.g., serving as a duct for a catheter. These internal inserts may be made from a material with a shape-memory effect, e.g., Nitinol, and may be substantially more rigid than any other portion of the device. The internal inserts and/or structural filaments may be positioned at the edge of the interlaced portion so as to provide a relatively stiff edge and include NiTi, Platinum, and/or Tantalum. These internal inserts can also be positioned away, e.g., 1 mm, 2 mm, 3 mm, 4 mm, 5 mm, 10 mm, 20 mm, 30 mm, 40 mm, 50 mm, 60 mm, 70 mm, or 80 mm, from the edge of the interlaced portion. This positioning can provide a relatively flexible edge that can be capable of functioning as a gasket.

In the devices of the invention, the loops may be twisted, e.g., external members, and/or the loops may be formed and/or positioned at the edge of the interlaced portion. The loops may also be formed less than 80 mm, e.g., 1 mm, 2 mm, 3 mm, 4 mm, 5 mm, 10 mm, 20 mm, 30 mm, 40 mm, 50 mm, 60 min, 70 mm, or 80 mm, away from the edge of the interlaced portion, in some embodiments forming an external skeleton and a relatively flexible edge, e.g., an edge capable of functioning as a gasket. The loops may also be formed at the edge of the interlaced portion, but positioned and affixed at less than 80 mm, e.g., 1 mm, 2 mm, 3 mm, 4 mm, 5 mm, 10 mm, 20 mm, 30 mm, 40 mm, 50 mm, 60 mm, 70 mm, or 80 mm, away from the edge of the interlaced portion. These external members, e.g., loops, may be capable of providing structural support to the device. These loops may be formed by interlaced filaments, structural filaments, and/or internal inserts, and can be substantially more rigid than any other portion of the device. Any of these loops can be optionally attached by an adhesive component, a connecting structure, and/or soldered connection. Thus, the loops can be integrated with the remainder of the device. In other aspects of the invention, the loops may extend downward and/or upward from the horizontal plane of the device. In another aspect, a loop may be formed by at least two interlaced filaments, structural filaments, and/or internal inserts, and these interlaced filaments, structural filaments, and/or internal inserts may or may not re-enter the interlaced portion. In some embodiments, the device may also be a three-dimensional convex structure.

Any of the devices of the invention may taper at one or both ends. If the filaments are interlaced to form a filter, the openings within the filter can be big enough to let blood pass, but small enough to block emboli. In some embodiments, the devices may serve as a coronary stent, peripheral stent, a heart valve, and/or be combined with a synthetic graft.

In another aspect, the invention features methods of preventing passage of a particle from the aorta into the left subclavian, left common carotid, and/or brachiocephalic arteries by deploying into an aorta any of the above-described devices such that the device prevents a particle from passing to the left subclavian, left common carotid, and/or brachiocephalic arteries. In other aspects, the device features one or more loops (as described above) extending downward from the horizontal plane of the deployed device such that one or more of the loops contacts, e.g., a medial surface of the ascending aorta, one or more of the loops extending upwards from the deployed device such to contact a medial surface of a subclavian artery, and/or the interlaced portion contacting the ascending, descending aorta, or both.

As used herein, the term “interlaced” refers to any filament segments that are braided, woven, knitted, or twisted together.

As used herein, the term “relatively flexible” refers to any portion that is more flexible relative to the internal portions of the device.

As used herein, the term “relatively stiff” refers to any portion that is more stiff relative to the internal portion of the device.

As used herein, the term “provide functionality” refers to a property that provides additional utility (e.g., connection loop, internal duct, or creates a seal with contacted tissue) beyond the mere structural properties of the filament.

As used herein, the term “provide structural support” refers to the property contributing to the shape, stiffness, and stability of the device.

As used herein, the term “structural filament” refers to a filament that is stiffer and/or thicker than the remaining filaments.

As used herein, the term “blood” refers to all or any of the following: red cells (erythrocytes), white cells (leukocytes), platelets (thrombocytes), and plasma.

As used herein, the term “filaments” refers to any elongated structure (e.g., cords, fibers, yarns, wires, cables, and threads) fabricated from any non-degradable material (e.g., polycarbonate, polytetrafluorothylene (PTFE), expanded polytetrafluorothylene (ePTFE), polyvinylidene fluoride, (PVDF), polypropylene, porous urethane, Nitinol, fluoropolymers (Teflon®), cobalt chromium alloys (CoCr), and para-aramid (Kevlar®), or textile (e.g., nylon, polyester (Dacron®), or silk).

As used herein, the term “internal insert” refers to one or more additional filaments inserted into an interlaced portion. Portions and/or filaments of these one or more additional filaments may be interlaced with each other, they may be hollow, they may provide structural support, and may facilitate insertion, deployment, and retrieval of the device.

As used herein, the term “delivery cable” refers to any delivery system used in interventional cardiology to introduce foreign bodies to a treatment site (e.g., catheters, guidewires, tubes, and wires).

As used herein, the term “internal skeleton” refers to a structural element within the perimeter of the filter that provides structural support to the interior of the device.

### BRIEF DESCRIPTION OF THE DRAWINGS

The invention herein described, by way of example only, with reference to the accompanying drawings, wherein:

FIG. 1 is a schematic diagram of an edge of an interlaced portion of a device with a single filament internal insert located at the edge of the interlaced portion, where the internal insert exits and re-enters the interlaced portion to form a loop.

FIG. 2 is a schematic diagram of an edge of the interlaced portion of a device with an internal insert located at the edge, where the internal insert is more than one filament and exits and re-enters the interlaced portion to form a loop.

FIG. 3 is a schematic diagram of an edge of an interlaced portion of a device with an internal insert located at the edge, where the internal insert is three filaments. Here, one filament exits and re-enters the interlaced portion to form a loop. The loop is also twisted to provide increased stiffness.

FIG. 4 is a schematic diagram of device (11) with two external members (20 and 21) twisted together.

FIG. 5 is a schematic diagram of an external loop (2) that is formed at the edge of filtering portion (1). Loop (3) represents a configuration where loop (2) is moved or bent inward, towards the center of the interlaced portion (1).

FIG. 6 is a schematic diagram of the device where the external loops (4) are formed from internal insert (5) and arranged to form a passage, e.g., for guiding a guidewire or catheter.

FIG. 7 is a schematic diagram of interlaced portion (1) with a thicker element (6) alternating between being an interlaced filament (9) and an insert (8).

FIG. 8 is a schematic diagram of an interlaced portion (1) with a thicker filament (10) interlaced so as to form an internal skeleton. Here, the edge (12) is more flexible than the internal portion.

FIG. 9 is a schematic diagram of device (11) including interlaced filament (13) that is thicker than the remaining filaments and that exits the interlaced portion (1) to form an external loop (13).

FIG. 10 is a schematic diagram of device (11) where the end (14) tapers.

FIG. 11 is a schematic diagram of device (11) including internal insert (15) located less than 80 mm, e.g., 1 mm, 2 mm, 5 mm, 8 mm, 10 mm, 15 mm, 20 mm, 30 mm, 40 mm, 50 mm, 60 mm, 70 mm, or 80 mm, away from device edge (16).

FIG. 12A is a photograph of a cross section of DFT wire.

FIG. 12B is a schematic diagram of a filter mesh containing DFT wire.

FIG. 12C is a photograph of a radiopacity bead and clamp element for use in the invention.

FIG. 13A is a schematic diagram showing filter meshes of the indicated pore sizes.

FIG. 13B is a schematic diagram showing perforated films with the indicated patterns, sizes, and densities of pores.

FIG. 13C is a schematic diagram showing a filter mesh with a combination of DFT (Drawn Filled Tubing) and Nitinol wires.

FIGS. 14A-14C are photographs showing a variety of mechanisms for connecting the device to a catheter or delivery cable.

FIG. 15 is a schematic diagram of a side view of a plunger for use in introducing devices of the invention into a subject, e.g., through a catheter.

### DETAILED DESCRIPTION OF THE PREFERRED EMBODIMENTS

In general, the invention features methods of constructing devices (e.g., intravascular devices) out of woven or braided filaments. The devices, in addition to containing surfaces constructed from woven or braided filaments, also contains structural and/or functional elements constructed out of filaments that are integrated into the woven or braided surfaces (i.e., internal inserts). These internal insert filaments can be themselves woven or braided into the surfaces or can be inserted into the weave or braid.

The devices of the invention feature an interlaced portion, including, e.g., interlaced filaments and internal inserts. This interlaced portion provides structure to the device and may serve as a filter. The devices of the invention can also feature external members that may be sections of the interlaced filaments, or internal inserts exiting from the interlaced portion. These interlaced filaments or internal inserts may or may not re-enter the interlaced portion. The use of the same interlaced filaments or internal inserts for both the internal portion and external members may permit the modification of the devices properties, e.g., modify stiffness, strength, or shape, without the use of external connective methods, e.g., adhesive, solder, or external connecting structures between the internal interlaced portion and the external members. These external members can provide additional structural support for the device and can facilitate the creation of a seal between the filter of the device and a blood vessel wall. Alternatively, the interlaced portion itself may create a seal against the blood vessel wall and exhibit a relatively flexible edge that may act as a gasket.

The device of the invention may include an interlaced portion and internal inserts that exit the interlaced portion to form a loop, e.g., an external member, and optionally re-enter the interlaced portion. The interlace portion may filter and/or deflect emboli or other large objects from entering protected secondary vessels, while the external member may facilitate device placement within the primary vessel. The device can also serve as coronary stent, peripheral stent, synthetic graft, heart valve, gastrointestinal stent, laryngeal stent, ureteral stent, tracheal implant, or transdermal implant. The device can also be an intravascular device for preventing particles from passing from a primary blood vessel (e.g., the aorta) to one or more secondary blood vessels (e.g., the left subclavian, left common carotid, and brachiocephalic artery). In general, the device may further be compatible with common delivery methods used in interventional cardiology (e.g., TAVI procedures). The device may be integrated into a delivery system. In other embodiments the device may be detachable from the delivery system. Upon deployment, the device may be positioned so as to contact the orifice of one or more secondary blood vessels in the primary blood vessel, e.g., in the aortic arch. In still other embodiments, the filaments may be arranged so that the edge of the device is relatively flexible and may serve as a gasket for improved device performance within a primary vessel.

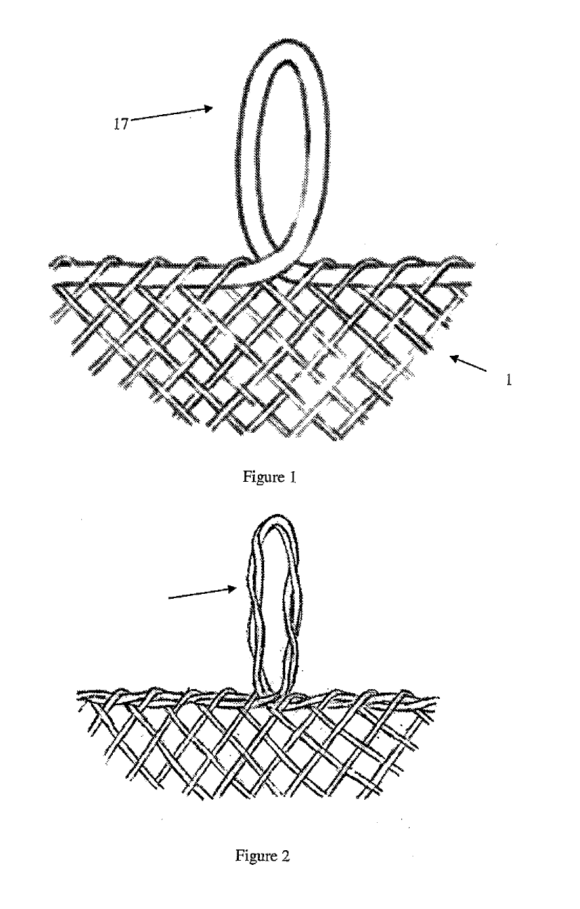

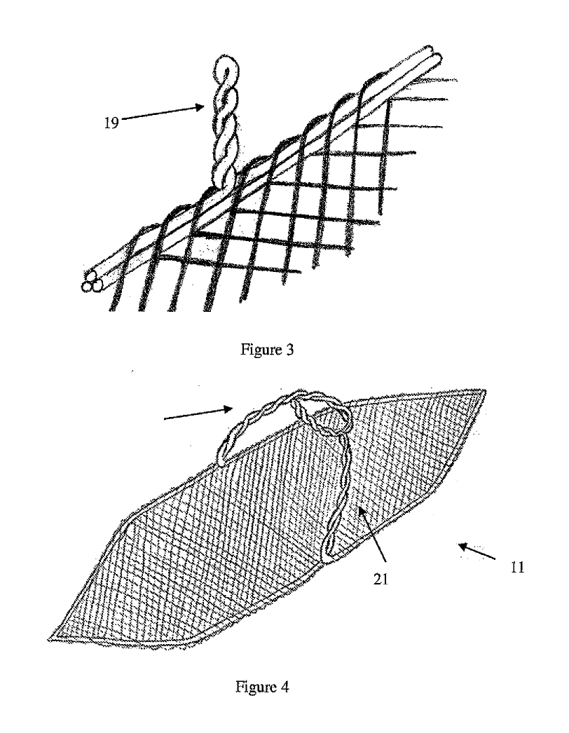

Reference is made to FIG. 1 and FIG. 2: FIG. 1 is a schematic diagram of internal insert (17) positioned at the edge of the interlaced portion, and FIG. 2 is a schematic diagram of internal insert (18) positioned at the edge of the interlaced portion. In both FIG. 1 and FIG. 2, the internal inserts (e.g., 17 and 18) exit the interlaced portion, form a loop, and then re-enter the interlaced portion. In some embodiments, the internal insert includes one filament, e.g., (17), while in other embodiments an internal insert (18) may include multiple filaments, e.g., 2, 3, 4, 5, 6, 7, 9, or 10 filaments. In embodiments where the internal insert includes multiple filaments, these multiple filaments may be interlaced together, e.g., (18), and/or interlaced within the interlaced portion. In some embodiments, a subsection of the internal insert filaments may exit the interlaced portion to form an external member (e.g., internal insert (19), as depicted in FIG. 3). In other embodiments the external loop may further twist to form an external member (e.g., internal insert (19), as depicted in FIG. 3).

Reference is made to FIG. 4: FIG. 4 is a schematic diagram of device (11) with two external members (20 and 21). In some embodiments, external inserts may be further interlaced both within the loop itself and with other external members. In these embodiments external members are connected, e.g., twist, loop, integrate, braid, or weave, with one or more, e.g., 2, 3, 4, 5, or 6 additional external members to form a substantial external member. The internal inserts may still include filaments from the interlaced portion that exit the interlaced portion, form the loop, and then re-enter the interlaced portion.

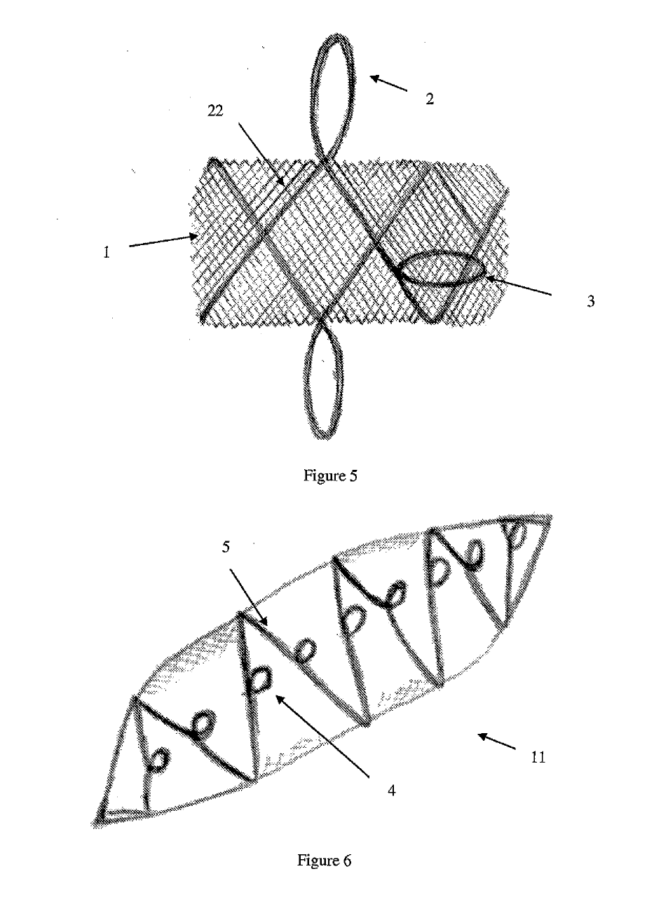

Reference is made to FIG. 5: FIG. 5 is a schematic diagram of interlaced portion (1) including structural filaments, e.g., a filament that is stiffer and/or thicker than the remaining filaments (22), and external members (e.g., 2 and 3). In some embodiments, structural filament (22) exits the interlaced portion, forms a loop at the edge the interlaced portion, and then re-enters the interlaced portion. This loop may subsequently be affixed at a position some distance away, e.g., 1 mm, 2 mm, 3 mm, 4 mm, or 5 mm away, from the edge of the interlace portion (e.g., (3)). In other embodiments, these external loops and members may provide additional functionality, e.g., act as a connection loop or duct. In other embodiments, these structural inserts may be replaced or combined with internal inserts. The internal inserts or structural filaments may be hollow so as to permit a guidewire and/or catheter to pass or may serve as a connection point for a deployment device.

Reference is made to FIG. 6: FIG. 6 is a schematic diagram of device (11) including structural filament (e.g., (5)), and external members (e.g., (4)). In some embodiments, the internal insert (e.g., (5)) is generally located some distance from the edge of device (11), thereby providing a relatively flexible edge. In other embodiments, the arrangement of the external members (e.g., (4)) provides additional functionality by creating a duct for a guide wire and/or catheter. This duct may also facilitate deployment and retrieval. In some embodiments, the external members may extend upwards and/or downwards from the horizontal plane of the device. These extending members may additional include two distinct supporting portions and a bend. In these embodiments, upon deployment, installation or release, an upward extending member may extend into an innominate artery. For example, a first support portion of this upper member may come into contact with a right internal wall of the innominate artery, the bend may come into contact with a right internal wall of the innominate artery and a second portion of the upper member may come into contact with a superior portion of a left internal wall of the innominate artery. The multiple possible contact or holding points of this upper member with the innominate artery may hold the device in place against a blood flow in the aorta, may prevent a roll of device within the aorta, and/or prevent the device from rising beyond a desired distance from an entry point of the innominate, left carotid, and left subclavian artery. This upper member may also prevent the device from sliding out of position in a direction of blood flow or of reverse flow in the aorta. This upper member may further exert a downward force on the device to counter a lift that may be exerted by any lower members, and to keep the device away from the entry points of the branch arteries of the aorta.

In some embodiments, the upper member may be inserted into, e.g., a left subclavian artery where a curve of the bend may be held against a left inner wall of the left subclavian artery, and a second portion may engage a counter wall.

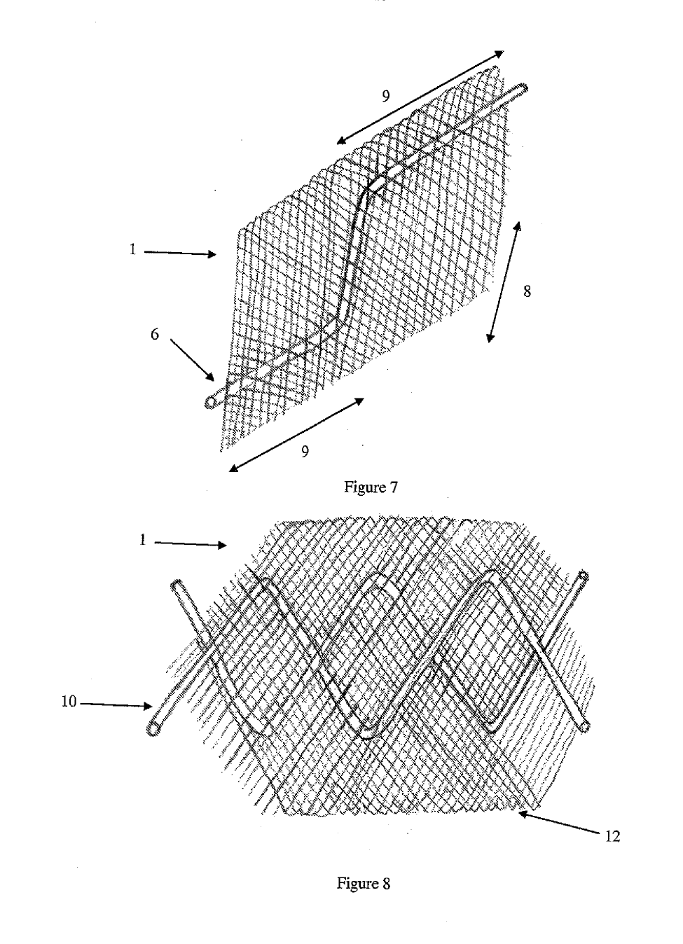

Reference is made to FIG. 7 and FIG. 8: FIG. 7 is a schematic diagram of the interlaced portion (1) including internal insert and/or structural filament (6), and FIG. 7 is a schematic diagram of interlaced portion (1) and internal insert and/or structural filament (10). In some embodiments, as depicted in FIG. 7, the internal insert and/or structural filament may alternate between interlaced within an interlaced portion (1), e.g., section (9), or simply inserted within the portion, e.g., section (8). In embodiments where the internal insert and/or structural filaments are located some distance from the edge (12) of the device, the internal insert and/or structural element (10) may form an internal skeleton. This internal skeleton may increase the strength, stability, and rigidity of the internal portion while the edge (12) may remain relatively flexible. In some embodiments, this substantial flexibility allows the edge (12) of the device to function as a gasket when inserted in a primary vessel. This gasket function may permit an enhanced seal when filtering particulate.

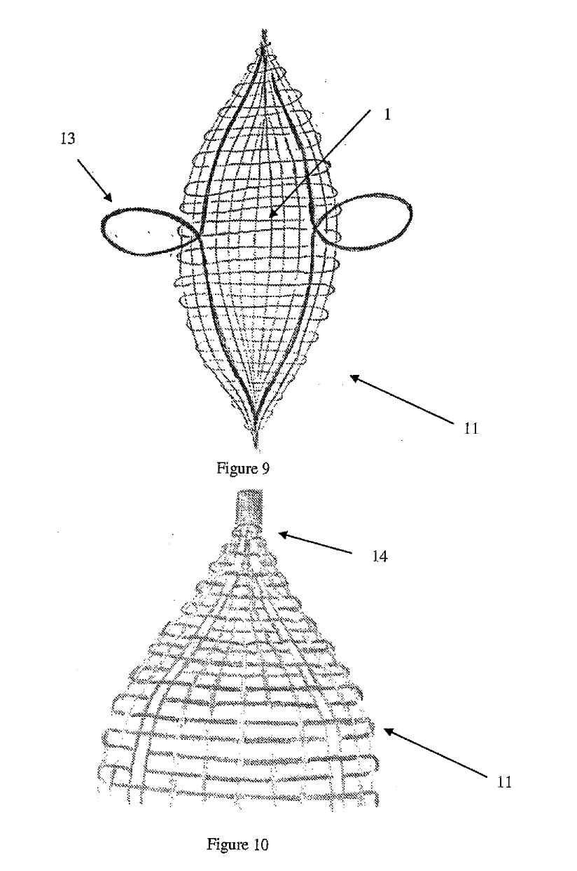

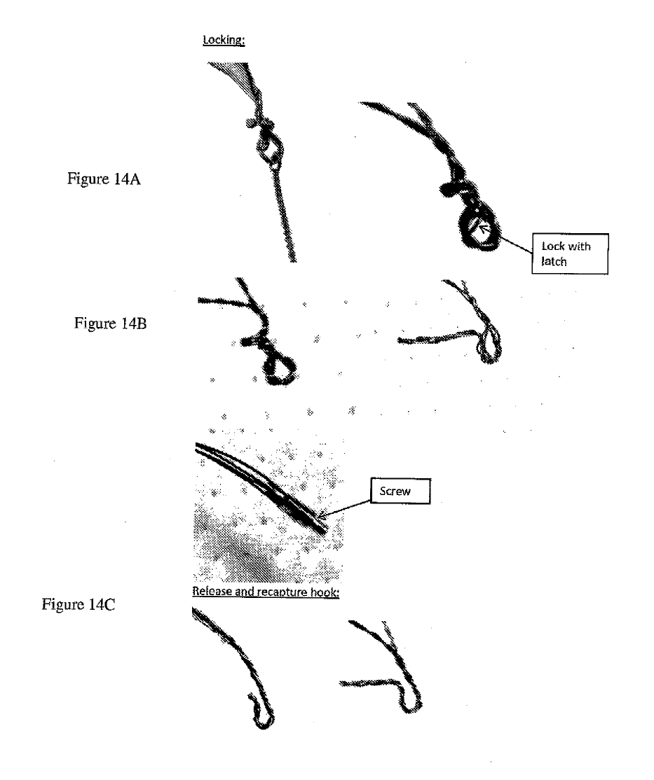

Reference is made to FIG. 9 and FIG. 10: FIG. 9 is a schematic diagram of device (11) with interlaced portion (1) and external member (13), and FIG. 10 is a schematic diagram of device (11) with tapered end (14). In some embodiments, interlaced filament (13) is stronger, e.g., thicker, stiffer, or NiTi, than the remaining filaments, and exits the interlaced portion (1) to form an external loop (13). This filament may be some distance from the edge of the device, permitting a relatively flexible edge. In some embodiments, the ends of device (11) taper as depicted in FIG. 10 (14). A deployment device may attach at a tapered end, e.g., via a mechanism shown in FIGS. 14A-14C.

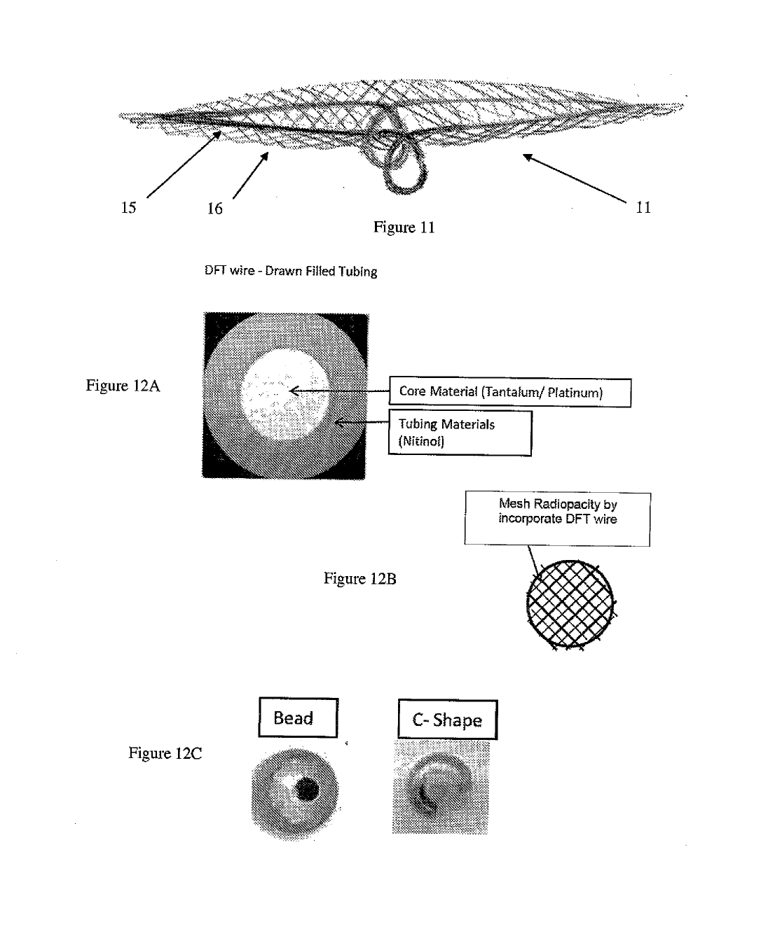

Reference is made to FIG. 11: FIG. 11 is a schematic diagram of device (11) including internal inserts (15) and a relatively flexible edge (16). In some embodiments, the interlaced portion within the internal insert perimeter may resemble a dome or a convex surface. This geometry may be modified.

In some embodiments, it is desirable to incorporate radiopaque elements into the intra-vascular device. Such radiopaque elements can be affixed to, or incorporated into the intra-vascular device (e.g., affixed to the internal inserts or interwoven filaments). The radiopaque element can be a bead or clamp (e.g., as depicted in FIG. 12C). In the case of a clamp, the element can be crimped onto the intra-vascular device. In any of the embodiments of the invention, radiopaque material can be incorporated into the filament forming the internal inserts or interwoven filaments of the intra-vascular device (see, e.g., FIG. 12B). For example, portions of the internal inserts or interlaced filaments can be constructed out of DFT wire. Such wire can contain, e.g., a core of tantalum and/or platinum and an outer material of, e.g., Nitinol (see, e.g., FIG. 12A).

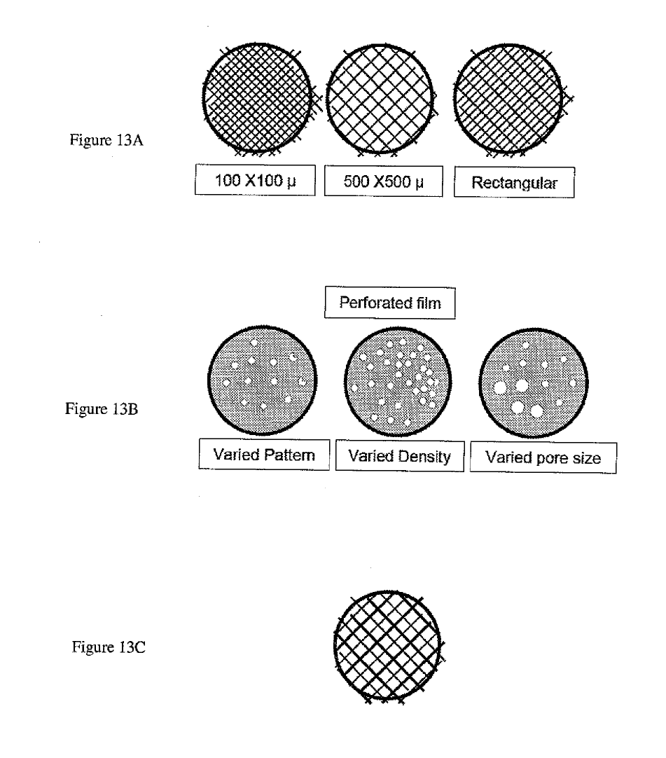

In some embodiments, interlaced portion (1) may include an additional fine wire netting or mesh, e.g., as depicted in FIGS. 13A and 13C, or perforated film, e.g., as depicted in FIG. 13B, such as a mesh or sheet having holes or porosity of 50-950 microns, e.g., 50, 150, 250, 350, 450, 550, 650, 750, 850, or 950 microns. The perforated film may be perforated prior to the inclusion with the device. The film may also be perforated post inclusion with the device (e.g., by laser drilling or electric sparks). In embodiments where a perforated film is present, the pores can have constant or varied pore patterns, constant or varied pore densities, and/or constant or varied pore sizes (FIG. 13B). The additional filters may be braided, weaved, clustered, knitted, or knotted. The additional filters may be a non-degradable material (e.g., polycarbonate, polytetrafluorothylene (PTFE), expanded polytetrafluorothylene (ePTFE), polyvinylidene fluoride, (PVDF), polypropylene, porous urethane, Nitinol, fluropolymers (Teflon®), cobalt chromium alloys (CoCr), and para-aramid (Kevlar®)), or textile (e.g., nylon, polyester (Dacron®), or silk). The filter may be a combination of materials (e.g., the combination of DFT and Nitinol wires as depicted in FIGS. 13A and 13B). The additional filters may also be coated with an anti-thrombogenic agent to prevent a thrombogenic reaction.

In some embodiments, one or more filaments or internal inserts may include a lumen, such as, for example, a hollow wire, which may hold, for example, a medicament that may be released into an artery or area where the device is implanted.

In some embodiments, device (11) may assume a substantially elliptical or elongated shape. Other shapes may be used. Because the aortic anatomy can vary between individuals, embodiments of the intra-vascular device of the invention are shaped to adapt to a variety of aortic anatomies. The size of the device (11) may be pre-sized and pre-formed to accommodate various patient groups (e.g., children and adults) or particular aortic anatomy. The device may vary in length from 10 mm to 120 mm (e.g., 25 mm, 45 mm, 60 mm, 75 mm, 90 mm, or 105 mm) and width from 5 mm to 70 mm (e.g., 10 mm, 20 mm, 30 mm, 40 mm, 50 mm, or 60 mm).

In certain embodiments, the stiffness of the intra-vascular device will be determined by the stiffness of interwoven filaments, internal inserts, and/or structural filaments. For example, the device can be stiffened by the inclusion of heavier gauge wire or by the inclusion of stiffer internal inserts. Furthermore, multiple wires of a certain gauge can be wound together to increase the stiffness of the device (e.g., the device can include 2, 3, 4, 5, or more filaments to increase the stiffness of the intra-vascular device).

Reference is made to FIGS. 14A-14C. As described above, a variety of configurations can be used to connect the intra-vascular filter to a plunger (e.g., a plunger connected with a delivery cable disposed within a catheter). FIG. 14A depicts a locking mechanism with a latch. FIG. 14B depicts a screw whereby the intra-vascular device can be mated with a screw on a plunger. FIG. 14C depicts a release and recapture hook for connecting the intra-vascular device with a plunger. In some embodiments, a hook may include a latch or wire strand that may be part of the device.

In other embodiments, the device (11), catheter, or delivery cable may end in a loop and may be threaded through a latch. When so threaded, a wire or catheter fitted with a looped end may be clicked into a hook and may securely push the device into place or pull the device out of position from a blood vessel (e.g., the aorta).

In some embodiments, the hook may end in a ball-tip so that strands from the device (11) do not fray or scratch the vessel wall or the inner tube of a catheter.

In other embodiments, a clasp at an end of the device may be pressed into or onto a clasp at, for example, an end of a catheter or delivery cable, and the two clasps may be joined by such pressing. In some embodiments, the device may be rotated clockwise or counter-clockwise respectively.

In an installed position, the intra-vascular device may be inserted into a first blood vessel. In some embodiments, the first blood vessel may be or include an aorta, though the device may be inserted into other vessels. The interlaced portion (1) of the device may be positioned so that an opening of a second blood vessel is covered by the filter, so that, for example, large particles are filtered, blocked, or deflected from entering, for example, the left subclavian, left common carotid, or brachiocephalic artery, or any combination thereof (e.g., the left subclavian, left common carotid, and brachiocephalic artery; the left subclavian and left common carotid artery; left common carotid and brachiocephalic artery; and the left common carotid and brachiocephalic artery). The space under interlaced portion (1) may allow unfiltered blood to pass by the branch artery of the aorta. The space in the aorta that is left below the filter means that not all blood passing through the aorta is subject to the filtering or deflecting process of interlaced portion (1). In an installed position, the device remains substantially flat (e.g., does not exceed a radius of curvature of 80 mm).

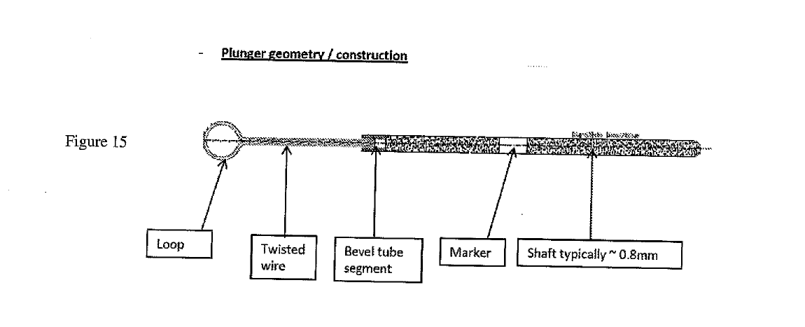

Reference is made to FIG. 15. A shaft or plunger for use in connection with the device can terminate in a loop (as depicted in FIG. 15) or a screw. In embodiments where a loop is present, the loop can be generated by winding two wires together leaving a loop at the distal end (FIG. 15). The shaft or plunger can, e.g., include a radiopaque element. Furthermore, the shaft or plunger can feature a rectilinear (e.g., square) or curved (e.g., oval or circular) cross section. Differences in cross sectional shape can have advantageous properties with respect to controlling the positioning of the intra-vascular device within the aorta. In certain embodiments, a shaft or plunger may replace or be used in conjunction with a delivery cable.

In still other embodiments, device (11) may be adapted for use with other embolism protection devices (e.g., those described U.S. application Ser. Nos. 13/300,936, and 13/205,255; in U.S. Publication Nos. 2008-0255603 and 2011-0106137; and in U.S. Pat. Nos. 8,062,324 and 7,232,453), each of which is hereby incorporated by reference in its entirety.

All publications and patents cited in this specification are incorporated herein by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A three-dimensional device comprising an interlaced portion and at least one loop portion, wherein:
a. said interlaced portion comprises a plurality of filaments interlaced together;
b. at least one of said interlaced filaments exits and re-enters the interlaced portion, thereby forming one or more loops; and
c. said interlaced portion includes an edge around the perimeter of said interlaced portion.

2. The device as in claim 1, wherein said one or more loops twist to form an external member.

3. The device as in claim 1 or 2, wherein at least two interlaced filaments exit the interlaced portion, combine to form one or more loops, and re-enter the interlaced portion.

4. The device as in claim 1 or 2, wherein at least two interlaced filaments exit the interlaced portion, combine to form a one or more loops, and do not re-enter the interlaced portion.

5. The device as in claim 3 or 4, wherein said one or more loops are formed by filaments that are structural filaments.

6. The device as in any one of claims 1-5, further comprising internal inserts.

7. The device as in claim 6, wherein said at least one of said internal inserts exits and re-enters the interlaced portion, thereby forming said one or more loops.

8. The device as in claim 6 or 7, wherein at least two internal inserts exit the interlaced portion, combine to form said one or more loops, and re-enter the interlaced portion.

9. The device as in claim 6 or 7, wherein at least two interlaced filaments exit the interlaced portion, combine to form said one or more loops, and do not re-enter the interlaced portion.

10. The device as in any one of claims 6-9, wherein at least one internal insert remains within the interlaced portion, thereby maintaining axial strength.

11. The device as in any one of claims 6-10, wherein said internal inserts are capable of providing structural support.

12. The device as in any one of claims 6-11, wherein said internal inserts are capable of providing functionality.

13. The device as in any one of claims 6-12, wherein said internal inserts are made from a material with a shape-memory effect.

14. The device as in any one of claims 6-13, wherein said internal inserts are positioned at the edge of the interlaced portion, wherein said internal inserts provide a relatively stiff edge.

15. The device as in claim 6-14, wherein said internal inserts are positioned less than 80 mm from the edge of the interlaced portion, thereby providing a relatively flexible edge to the filtering section.

16. The device as in claim 15, wherein said relatively flexible edge is capable of functioning as a gasket.

17. The device as in any one of claims 6-16, wherein said internal inserts are substantially more rigid than any other portion of the device.

18. The device as in any one of claims 1-17, wherein said filaments comprise NiTi, Platinum, and/or Tantalum.

19. The device as in any one of claims 6-18, wherein said internal inserts comprise NiTi, Platinum, and/or Tantalum.

20. The device as in any one of claims 1-19, wherein said filaments comprise NiTi.

21. The device as in any one of claims 6-20, wherein said internal inserts comprise NiTi.

22. The device as in any one of claims 1-21, wherein said one or more loops are formed at said edge of the interlaced portion, thereby forming an external member.

23. The device as in any one of claims 1-22, wherein said one or more loops are formed at the edge of the interlaced portion, wherein said one or more loops are capable of providing functionality.

24. The device as in any one of claims 1-23, wherein said one or more loops are formed less than 80 mm from the edge of the interlaced portion, thereby forming an external skeleton and a relatively flexible edge.

25. The device as in any one of claims 1-24, wherein said one or more loops are formed at the edge of said interlaced portion and affixed at less than 80 mm from the edge of said interlace portion, thereby forming an external member.

26. The device as in claim 25, wherein said external member is capable of providing structural support.

27. The device as in any one of claims 1-26, wherein said device tapers at one or both ends.

28. The device as in any one of claims 1-27, wherein said one or more loops twist to form an external member.

29. The device as in any one of claims 1-27, wherein said one or more loops twist to form an external member.

30. The device as in any one of claims 1-29, wherein said device is configured to reside in a living body.

31. An intra-vascular device comprising a filter, wherein:
a. said filter comprises a three-dimensional structure comprising an interlaced portion and one or more loop portions, wherein:
i. said interlaced portion comprises a plurality of filaments interlaced together and patterned to form a filter;
ii. said interlaced filaments exits and re-enters the interlaced portion, thereby forming one or more loops;
iii. one or more loop of said portions extends downward from the horizontal plane; and
iv. one or more loop of said portions extends upwards from the horizontal plane.

32. An intra-vascular device comprising a filter, wherein:
a. said filter comprises a three-dimensional structure comprising an interlaced portion, one or more internal insert filaments and/or structural filaments, and one or more loop portions, wherein:
i. said interlaced portion comprises a plurality of filaments interlaced together and patterned to form a filter;
ii. at least one of said interlaced filaments, structural filaments, and/or internal inserts exits and re-enters the interlaced portion, thereby forming one or more loops;
iii. one or more loop of said portions extends downward from the horizontal plane; and
iv. one or more loop of said portions extends upwards from the horizontal plane.

33. The device as in of claim 31 or 32, wherein said device is a three-dimensional convex structure, and wherein said interlaced filaments, structural filaments, and/or internal inserts form one or more loops and said one or more loops are not attached by an adhesive component or connecting structure.

34. The device as in any one of claims 31-33, wherein said one or more loops are formed by structural filaments.

35. The device as in any one of claims 31-33, wherein said one or more loops are formed by interlaced filaments.

36. The device as in any one of claims 31-33, wherein said one or more loops are formed by internal inserts.

37. The device as in any one of claims 31-36, wherein the openings within said filter are big enough to let blood pass, but small enough to block emboli.

38. The device as in claim 37, wherein said interlaced portion and said one or more loops are integrated.

39. The device as in claim 38, wherein said integration is not via an adhered or soldered connection.

40. The device as in claim 39, wherein said one or more loops twist to form an external member.

41. The device as in claim 40, wherein said external member is substantially more rigid than any other portion.

42. The device as in any one of claims 31-41, wherein at least two interlaced filaments exit the interlaced portion, combine to form a single loop, and re-enter the interlaced portion.

43. The device as in any one of claims 31-41, wherein at least two interlaced filaments, structural filaments, and/or internal insert filaments exit the interlaced portion, combine to form a single loop, and re-enter the interlaced portion.

44. The device as in any one of claims 31-41, wherein at least two interlaced, structural filaments, and/or internal insert filaments combine to form a single loop, and do not re-enter the interlaced portion.

45. The device as any of claims 31-44, wherein said filaments are made from a material with a shape-memory effect.

46. The device as in any one of claims 31-45, wherein said filaments are substantially more rigid than any other portion or are made from a material with a shape-memory effect.

47. The device as in any one of claims 32-46, wherein said internal inserts comprise a single wire.

48. The device as in any one of claims 32-46, wherein said internal inserts comprise two or more wires.

49. The device as in any one of claim 32-48, wherein said internal inserts comprise at least one filament.

50. The device as in any one of claims 32-49, wherein said internal inserts are capable of providing structural support.

51. The device as in any one of claims 32-50, wherein said internal inserts are made from a material with a shape-memory effect.

52. The device as in any one of claims 31-51, wherein said filaments comprise NiTi.

53. The device as in any one of claims 31-52, wherein said filaments comprise NiTi, Platinum, and Tantalum.

54. The device as in any one of claims 31-52, wherein said filaments comprise NiTi, Platinum, or Tantalum.

55. The device as in any one of claims 1-54, wherein said device is capable of serving as a coronary stent.

56. The device as in any one of claims 1-54, wherein said device is capable of serving as a peripheral stent.

57. The device as in any one of claims 1-54, wherein said device is capable of combination with synthetic grafts.

58. The device as in any one of claims 1-54, wherein said device is incorporated into an artificial heart valve.

59. A method of preventing passage of a particle from the aorta into the left subclavian, left common carotid, or brachiocephalic arteries comprising deploying the device of any of claims 1-54 in said aorta such that said device prevents particles from passing to the left subclavian, left common carotid, or brachiocephalic arteries.

60. A method of preventing passage of a particle from the aorta into a subclavian artery comprising deploying the device as in any one of claims 1-54 in said aorta such that:
a. said one or more loops configured to extend downward from said deployed device contacts a medial surface of the ascending aorta;
b. said one or more loops configured to extend upward from said deployed device contacts a medial surface of a subclavian artery;
c. said interlaced portion contacts both the ascending and descending aorta;
wherein, said deployment of said device prevents passage of particles from said aorta into said subclavian artery.
